(19) United States
(12) Patent Application Publication
(10) Pub. No.: US 2017/0265866 A1
(43) Pub. Date: Sep. 21, 2017

(54) TARGETING SYSTEMS FOR PROVIDING ACCURATE PLACEMENT OF MAGNETIC ANASTOMOSIS DEVICES

(71) Applicant: GI Windows, Inc., West Bridgewater, MA (US)

(72) Inventors: Marvin Ryou, Melrose, MA (US); Christopher Thompson, Needham, MA (US); James Wright, West Bridgewater, MA (US); Peter Lukin, Norfolk, MA (US)

(21) Appl. No.: 15/461,376

(22) Filed: Mar. 16, 2017

Related U.S. Application Data
(60) Provisional application No. 62/309,235, filed on Mar. 16, 2016.

(51) Int. Cl. A61B 17/11; A61B 1/018; A61B 6/03; A61B 6/00; A61B 8/08; A61B 6/12

(52) U.S. Cl. CPC A61B 17/1114; A61B 17/11; A61B 8/0841; A61B 6/12; A61B 6/032; A61B 6/487; A61B 1/018; A61B 2017/00876

(57) ABSTRACT

The invention provides a system for providing improved placement of magnetic compression devices at a desired target site so as to create anastomoses between tissues. The system includes a targeted member or medium configured to be placed within a hollow body of a patient. The hollow body may include, but is not limited to, the stomach, gallbladder, pancreas, duodenum, small intestine, large intestine, bowel, vasculature, including veins and arteries, or the like. The targeted member or medium is configured to provide a target site at desired anatomical location within the hollow body for formation of an anastomosis between a first portion of tissue of the hollow body at the target site and a second portion of tissue of the hollow body.

### CROSS-REFERENCE TO RELATED APPLICATIONS

This application claims the benefit of, and priority to, U.S. Provisional Application Ser. No. 62/309,235, filed Mar. 16, 2016, the content of which is incorporated by reference herein in its entirety.

### FIELD OF THE INVENTION

The invention relates to deployable magnetic compression devices, and, more particularly, to a system for providing targeted placement of magnetic anastomosis devices at a desired site so as to improve the accuracy of anastomoses creation between tissues, organs, or the like.

### BACKGROUND

Bypasses of the gastroenterological (GI), cardiovascular, or urological systems are typically formed by cutting holes in tissues at two locations and joining the holes with sutures or staples. A bypass is typically placed to route fluids (e.g., blood, nutrients) between healthier portions of the system, while bypassing diseases or malfunctioning tissues. The procedure is typically invasive, and subjects a patient to risks such as bleeding, infection, pain, and adverse reaction to anesthesia. Additionally, a bypass created with sutures or staples can be complicated by post-operative leaks and adhesions. Leaks may result in infection or sepsis, while adhesions can result in complications such as bowel strangulation and obstruction. While traditional bypass procedures can be completed with an endoscope, laparoscope, or robot, it can be time consuming to join the holes cut into the tissues. Furthermore, such procedures require specialized expertise and equipment that is not available at many surgical facilities.

As an alternative to sutures or staples, surgeons can use mechanical couplings or magnets to create a compressive anastomosis between tissues. For example, compressive couplings or paired magnets can be delivered to tissues to be joined. Because of the strong compression, the tissue trapped between the couplings or magnets is cut off from its blood supply. Under these conditions, the tissue becomes necrotic and degenerates, and at the same time, new tissue grows around points of compression, e.g., on the edges of the coupling. With time, the coupling can be removed, leaving a healed anastomosis between the tissues.

Nonetheless, the difficulty of placing the magnets or couplings limits the locations that compressive anastomosis can be used. In most cases, the magnets or couplings have to be delivered as two separate assemblies, requiring either an open surgical field or a bulky delivery device. For example, existing magnetic compression devices are limited to structures small enough to be deployed with a delivery conduit e.g., an endoscopic instrument channel or laparoscopic port. When these smaller structures are used, the formed anastomosis is small and suffers from short-term patency. Furthermore, placement of the magnets or couplings can be imprecise, which can lead to anastomosis formation in locations that is undesirable or inaccurate.

Thus, there still remains a clinical need for reliable devices and minimally-invasive procedures that facilitate compression anastomosis formation between tissues in the human body.

### SUMMARY

The invention provides improved devices and techniques for minimally-invasive formation of anastomoses within the body, e.g., the gastrointestinal tract. Such devices and techniques facilitate faster and less-expensive treatments for chronic diseases such as obesity and diabetes. Such techniques also reduce the time and pain associated with palliative treatments for diseases such as cancers, such as stomach or colon cancer.

More specifically, the invention provides a system for providing improved placement of magnetic compression devices at a desired target site so as to create anastomoses between tissues. The system generally includes a targeted member or medium configured to be placed within a hollow body of a patient. The hollow body may include, but is not limited to, the stomach, gallbladder, pancreas, duodenum, small intestine, large intestine, bowel, vasculature, including veins and arteries, or the like. The targeted member or medium is configured to provide a target site at desired anatomical location within the hollow body for formation of an anastomosis between a first portion of tissue of the hollow body at the target site and a second portion of tissue of the hollow body.

For example, if the hollow body is a bowel of the patient, the first portion may be a distal portion of the bowel and the second portion may be a proximal portion of the bowel. The bowel includes any segment of the alimentary canal extending from the pyloric sphincter of the stomach to the anus. In some embodiments, an anastomosis is formed to bypass diseased, malformed, or dysfunctional tissues. In some embodiments, an anastomosis is formed to alter the “normal” digestive process in an effort to diminish or prevent other diseases, such as diabetes, hypertension, autoimmune, or musculoskeletal disease. It should be noted that the system may be used for the formation of an anastomosis between a first portion of tissue of the hollow body at the target site and an adjacent tissue of a second hollow body (e.g., portal between the stomach and the gallbladder or the duodenum and the gallbladder, etc.).

The system further includes an access device configured to provide access between the first and second portions of tissue of the hollow body and further deliver and position first and second implantable magnetic anastomosis devices relative to the first and second portions of tissue or adjacent tissue for the formation of an anastomosis between tissues at the target site. The first and second implantable magnetic anastomosis devices are configured to be magnetically attracted to one another through a defined tissue area of the combined thickness of a wall of the tissues at the target site and exert compressive forces on the defined area to form the anastomosis.

In some embodiments, the system further includes an imaging modality configured to provide a visual depiction of the targeted member or medium within the hollow body and the surrounding anatomical location to thereby provide visual depiction of the location of the target site to an operator. For example, during a procedure, the imaging modality is configured to detect the targeted member or medium within the hollow body and further provide a visual display of the hollow body and the targeted member or medium within, so as to illustrate the targeting site. For example, the targeted member or medium may include a contrast material or agent configured to enhance the contrast of the targeting medium relative to surrounding tissue of the hollow body when viewed under a medical imaging procedure provided by the imaging modality to thereby enhance the visibility of the target site. The medical imaging procedure may include, but is not limited to, ultrasound (US), wavelength detection, X-ray-based imaging, illumination, computed tomography (CT), radiography, and fluoroscopy, or a combination thereof. Thus, the contrast material or agent may include radiopaque material or radiocontrast medium or agent, respectively. In some embodiments, the contrast material or agent may include a fluorescent tracer material, such as fluorescein, for example and the wavelength emission of fluorescein may be sensed by the imaging modality.

In some embodiments, the targeted member may include a balloon catheter, such that, upon advancing the catheter within the hollow body, the balloon can be expanded at the desired anatomical location to provide a target site indicating where the anastomosis should be formed. In some embodiments, the balloon member may include a radiocontrast medium configured to enhance the contrast of the expanded balloon member relative to surrounding tissue of the hollow body when viewed under an imaging procedure of the imaging modality.

In some embodiments, the balloon member may include a probe configured to emit a signal, including, but not limited to, radio waves, infrared radiation, visible light, or other communication. The imaging modality, or the access device, may be configured to detect such emissions so as to further provide indication as to the location of the target site. For example, in some embodiments, the access device may include a detection system configured to detect the targeted member, including detecting a signal emitted from the probe. For example, in the event that the balloon member includes a contrast material or agent, such as a fluorescent tracer material (e.g., fluorescein), the detection system of the access device may include a wavelength detection means configured to detect the wavelength emission of the fluorescein, thereby providing indication to an operator (e.g., surgeon) that the access device is within the desired target site and placement of the anastomosis devices can begin. In other embodiments, the detection system may be configured to detect the probe emissions.

The balloon member, when expanded at the desired anatomical location, may further serve as an anchor so as to assist in the placement of the magnetic anastomosis devices. For example, the access device may generally be embodied as an endoscope having a needle for the delivery of the devices. When the endoscope is in position (i.e., when placed adjacent to the target site), the needle of the endoscope may then pierce the tissues so as to provide access from the first and second portions of tissue of the hollow body (or between adjacent tissue of a second hollow body). When piercing through the tissues, the tip of the needle may generally make contact with the balloon member, thereby further providing tactile feedback to the operator that the needle puncture, and subsequent placement of the magnetic devices, is at the target site so as to provide further confirmation that anastomosis will occur at the desired location. The needle may then be used to deliver the first magnetic anastomosis device into the first portion of tissue and the second magnetic anastomosis device into the second portion of tissue. Accordingly, the first and second implantable magnetic anastomosis devices then become magnetically attracted to one another through a defined tissue area of the combined thickness of a wall of the tissues at the target site and exert compressive forces on the defined area to form the anastomosis.

Accordingly, the targeting system of the present disclosure provides improved placement of magnetic compression devices at a desired target site so as to create accurate anastomoses between tissues. By providing a targeted member or medium, an operator can better visualize the placement of magnetic compression devices within one or more hollow bodies of a patient and further ensure that such placement is more accurate, leading to anastomosis formation in the desired location, cutting down on repeat procedures. Furthermore, placement of the magnetic devices is achieved by way of a single scope, rather than having to work with two separate scopes, thereby overcoming the challenges encountered when dealing with two or more scope devices and further requiring less towers to support the additional scope(s) or components, decreasing costs and space required.

### BRIEF DESCRIPTION OF THE DRAWINGS

Features and advantages of the claimed subject matter will be apparent from the following detailed description of embodiments consistent therewith, which description should be considered with reference to the accompanying drawings, wherein:

FIG. 1 is a schematic illustration of a targeting system consistent with the present disclosure;

FIG. 2 shows several potential anatomical targets for anastomosis formation, where arrow A is stomach to small intestine, arrow B is small intestine to large intestine, arrow C is small intestine to small intestine, arrow D is large intestine to large intestine, and arrow E is stomach to large intestine;

FIG. 3 shows an exemplary magnetic anastomosis device delivered through an endoscope instrument channel such that the individual magnet segments self-assemble into a larger magnetic structure—in this particular case, an octagon;

FIG. 4A depicts two magnetic anastomosis devices attracting each other through tissue. As shown, the devices each comprise eight magnetic segments, however alternate configurations are possible. Once the two devices mate, the tissue that is trapped between the devices will necrose, causing an anastomosis to form. Alternatively, the tissue bound by the devices may be perforated after the devices mate to create an immediate anastomosis;

FIG. 4B shows the two magnetic anastomosis devices coupled together by magnetic attraction, capturing the intervening tissue. In some instances, the endoscope can be used to cut through the circumscribed tissue;

FIG. 5 shows one embodiment of a procedure for forming anastomosis using the targeting system of the present disclosure, specifically using a targeting member (e.g., balloon catheter) for establishing a target site indicating the location in which the anastomosis is to be formed and a single endoscope to deliver the magnetic devices to tissues adjacent to the target site;

FIG. 6 is an enlarged view showing the placement of the balloon catheter within a first portion of a tissue of a hollow body and the advancement of the endoscope to a second portion of tissue of the hollow body adjacent to the target site established by the balloon catheter;

FIG. 7 is an enlarged view illustrating the endoscope providing access from the second portion to the first portion of tissues by way of a needle (i.e., piercing of tissues) so as to allow for the delivery and deployment of the magnetic devices; and

FIG. 8 shows the needle delivering a first magnetic device into a first portion of the hollow body at the target site, which is then followed by deployment to of a second magnetic device into a second portion of the hollow body adjacent to the target site.

For a thorough understanding of the present disclosure, reference should be made to the following detailed description, including the appended claims, in connection with the above-described drawings. Although the present disclosure is described in connection with exemplary embodiments, the disclosure is not intended to be limited to the specific forms set forth herein. It is understood that various omissions and substitutions of equivalents are contemplated as circumstances may suggest or render expedient.

### DETAILED DESCRIPTION

The invention provides a system for providing improved placement of magnetic compression devices at a desired target site so as to create anastomoses between tissues. The system generally includes a targeted member or medium configured to be placed within a hollow body of a patient and provide a target site at desired anatomical location within the hollow body for formation of an anastomosis between a first portion of tissue of the hollow body at the target site and a second portion of tissue of the hollow body. The system further includes an access device configured to provide access between the first and second portions of tissue of the hollow body and further deliver and position first and second implantable magnetic anastomosis devices relative to the first and second portions of tissue or adjacent tissue for the formation of an anastomosis between tissues at the target site. The first and second implantable magnetic anastomosis devices are configured to be magnetically attracted to one another through a defined tissue area of the combined thickness of a wall of the tissues at the target site and exert compressive forces on the defined area to form the anastomosis.

In some embodiments, the system further includes an imaging modality configured to provide a visual depiction of the targeted member or medium within the hollow body and the surrounding anatomical location to thereby provide visual depiction of the location of the target site to an operator. For example, during a procedure, the imaging modality is configured to detect the targeted member or medium within the hollow body and further provide a visual display of the hollow body and the targeted member or medium within, so as to illustrate the targeting site.

In some embodiments, the targeted member may include a balloon catheter, such that, upon advancing the catheter within the hollow body, the balloon can be expanded at the desired anatomical location to provide a target site indicating where the anastomosis should be formed. In some embodiments, the balloon member may include a radiocontrast medium configured to enhance the contrast of the expanded balloon member relative to surrounding tissue of the hollow body when viewed under an imaging procedure of the imaging modality.

In some embodiments, the balloon member may include a probe configured to emit a signal, including, but not limited to, radio waves, infrared radiation, visible light, or other communication. The imaging modality, or the access device, may be configured to detect such emissions so as to further provide indication as to the location of the target site. For example, in some embodiments, the access device may include a detection system configured to detect the targeted member, including detecting a signal emitted from the probe.

Accordingly, the targeting system of the present disclosure provides improved placement of magnetic compression devices at a desired target site so as to create accurate anastomoses between tissues. By providing a targeted member or medium, an operator can better visualize the placement of magnetic compression devices within one or more hollow bodies of a patient and further ensure that such placement is more accurate, leading to anastomosis formation in the desired location, cutting down on repeat procedures. Furthermore, placement of the magnetic devices is achieved by way of a single scope, rather than having to work with two separate scopes, thereby overcoming the challenges encountered when dealing with two or more scope devices and further requiring less towers to support the additional scope(s) or components, decreasing costs and space required.

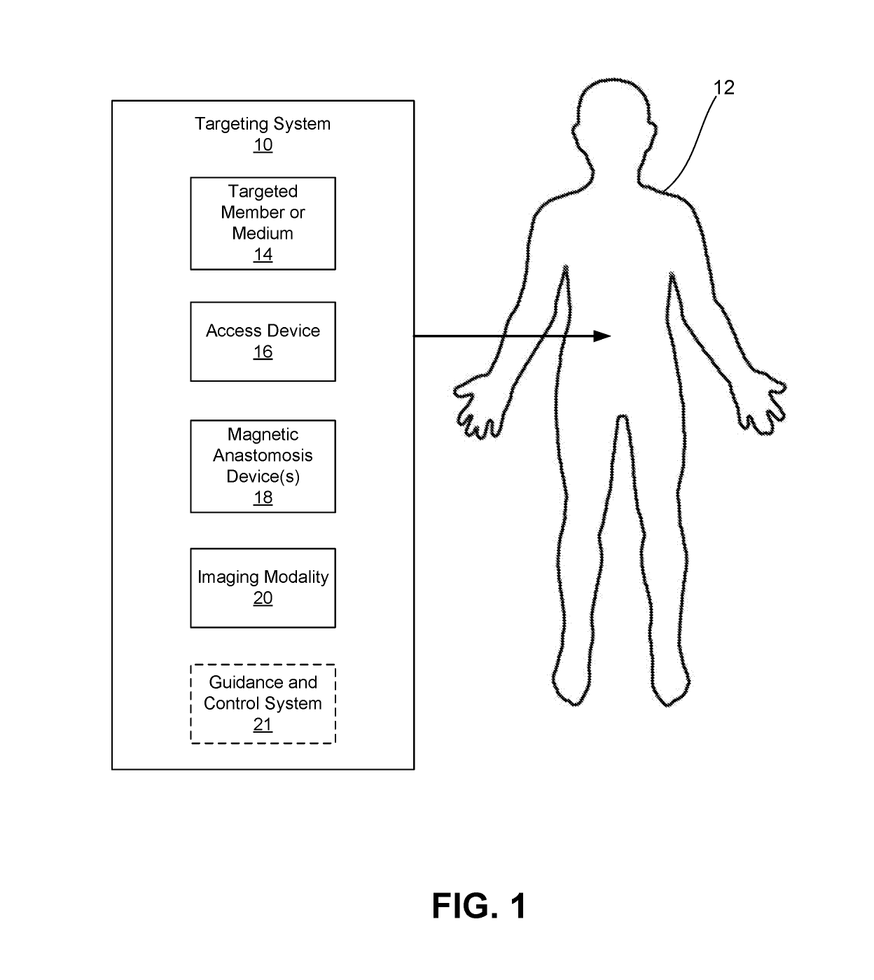

FIG. 1 is a schematic illustration of a targeting system 10 for providing targeted placement of magnetic anastomosis devices at a desired site so as to improve the accuracy of anastomoses creation between tissues within a patient 12. The targeting system 10 generally includes a targeted member or medium 14, an access device 16, magnetic anastomosis devices 18, and an imaging modality 20. In some embodiments, the targeting system 10 may further include a guidance and control system 21.

As will be described in greater detail herein, the guidance and control system 21 may be configured to sense, or otherwise locate, the targeted member or medium 14 and the access device 16 during a procedure and provide an operator (e.g., surgeon) a visual depiction of both the targeted member or medium 14 and access device 16 relative to one another so as to provide improved guidance of the access device 16 to the target site established by the targeted member or medium 14. For example, in some embodiments, the guidance and control system 21 may be configured to receive sensing input from both the access device 16 (e.g., sensors on the endoscope, such as ultrasound, video, images, etc.) and input from the imaging modality 20 and combine input from both so as to provide a more accurate display to the surgeon during a procedure.

As will be described in greater detail herein, the targeted member or medium 14 may include, for example, a contrast material or agent configured to enhance the contrast of the targeting member or medium relative to surrounding tissue of the hollow body when viewed under a medical imaging procedure provided by the imaging modality 20 so as enhance the visibility of the target site. For example, the medical imaging procedure may include, but is not limited to, ultrasound (US), wavelength detection, X-ray-based imaging, illumination, computed tomography (CT), radiography, and fluoroscopy, or a combination thereof. Thus, the contrast material or agent may include radiopaque material or radiocontrast medium or agent, respectively. In some embodiments, the targeted member may include a balloon catheter, for example, that may utilize a contrast material or agent to fill and expand the balloon and to further enhance the contrast of the targeting member relative to the surrounding tissue body when viewed under a medical imaging procedure. Additionally, or alternatively, the targeted member 14 may include a probe configured to emit signals for establishing the target site, wherein, upon detection of such signals via the imaging modality 20, or a detection system on the access device 16, an operator (e.g., surgeon) can confirm that the access device 16 is positioned adjacent to the target site and ensure accurate placement of the magnetic anastomosis devices 18.

The access device 16 may generally include a scope, including, but not limited to, an endoscope, laparoscope, catheter, trocar, or other delivery device. For most applications described herein, the access device 16 is an endoscope, including a delivery needle configured to deliver the magnetic anastomosis devices 18. Accordingly, the system 10 of the present disclosure relies on a single endoscope 16 for the delivery of the two magnetic devices 18. As will be described in greater detail herein, a surgeon may advance the endoscope 16 within a hollow body of the patient 12 and position the endoscope 16 at the desired anatomical location for formation of the anastomosis based on either a visual depiction of the location of the target site as provided by the imaging modality 20 or based on one or more detected signals, or both. For example, the imaging modality 20 may include a display in which an image, or other visual depiction, is displayed to the surgeon illustrating the target site established by the targeted member or medium 14 and surrounding tissue. The surgeon may then rely on such a visual depiction when advancing the endoscope through the hollow body so as to position the hollow body at a portion of tissue adjacent to the other portion of tissue at the target site, thereby ensuring the placement of the magnetic devices 18 is accurate.

It should be noted that the hollow body through which the targeted member or medium 14 and the access device 16 may pass include, but is not limited to, the stomach, gallbladder, pancreas, duodenum, small intestine, large intestine, bowel, vasculature, including veins and arteries, or the like.

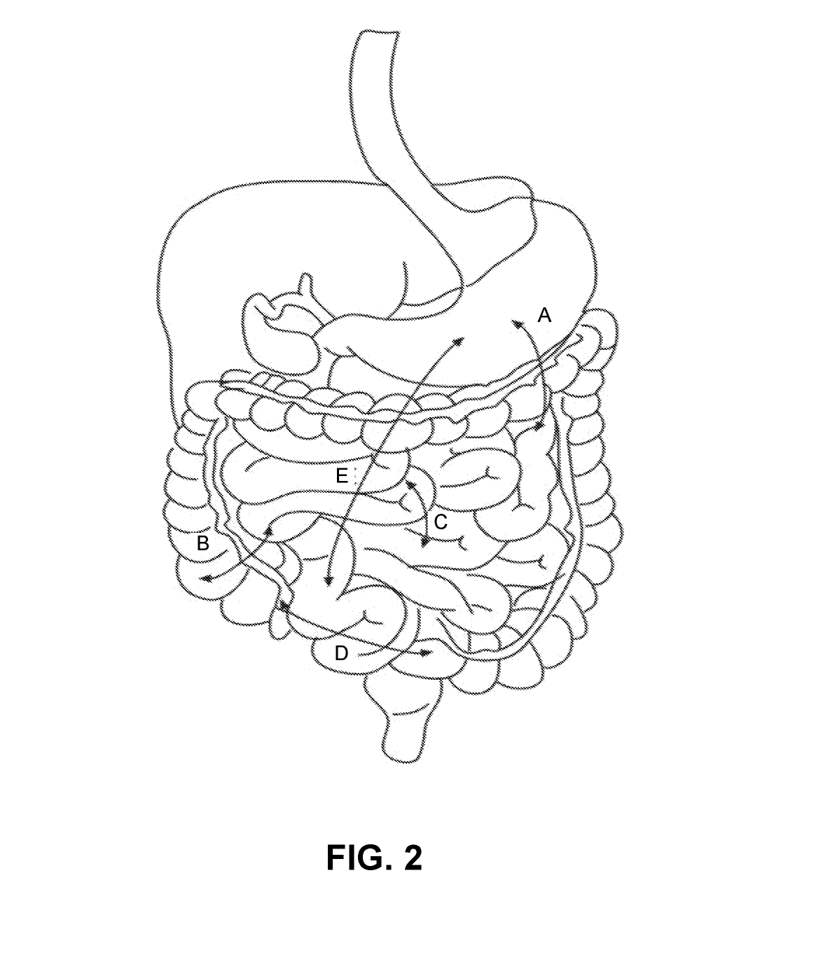

In some embodiments, the self-assembling magnetic devices are used to create a bypass in the gastrointestinal tract. Such bypasses can be used for the treatment of a cancerous obstruction, weight loss or bariatrics, or even treatment of diabetes and metabolic disease (i.e. metabolic surgery). FIG. 2 illustrates the variety of gastrointestinal anastomotic targets that may be addressed with the devices of the invention, such targets include stomach to small intestine (A), stomach to large intestine (E), small intestine to small intestine (C), small intestine to large intestine (B), and large intestine to large intestine (D). Accordingly, the invention provides improved devices and techniques for minimally-invasive formation of anastomoses within the body, e.g., the gastrointestinal tract. Such devices and techniques facilitate faster and less-expensive treatments for chronic diseases such as obesity and diabetes. Such techniques also reduce the time and pain associated with palliative treatments for diseases such as cancers, such as stomach or colon cancer.

For example, if the hollow body through which the targeted member or medium 14 and the access device 16 may pass is a bowel of the patient, the first portion may be a distal portion of the bowel and the second portion may be a proximal portion of the bowel. The bowel includes any segment of the alimentary canal extending from the pyloric sphincter of the stomach to the anus. In some embodiments, an anastomosis is formed to bypass diseased, malformed, or dysfunctional tissues. In some embodiments, an anastomosis is formed to alter the “normal” digestive process in an effort to diminish or prevent other diseases, such as diabetes, hypertension, autoimmune, or musculoskeletal disease. It should be noted that the system may be used for the formation of an anastomosis between a first portion of tissue of the hollow body at the target site and an adjacent tissue of a second hollow body (e.g., portal between the stomach and the gallbladder, the duodenum and the gallbladder, stomach to small intestine, small intestine to large intestine, stomach to large intestine, etc.).

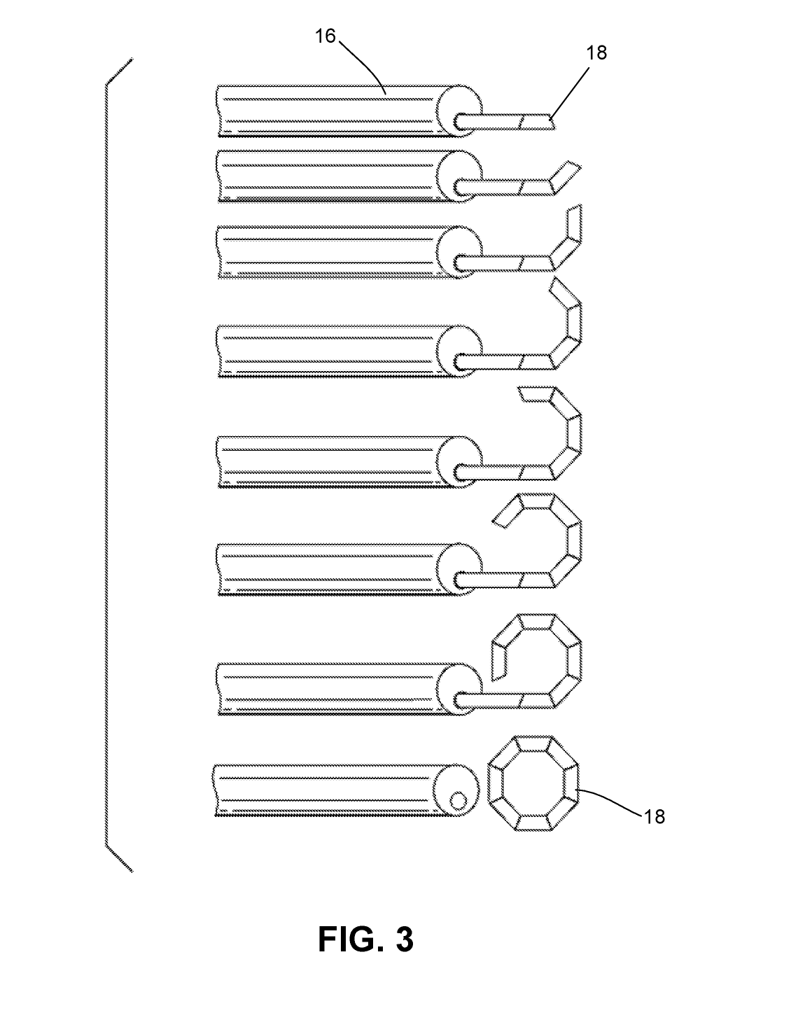

In an endoscopic procedure, the self-assembling magnetic devices can be delivered using a single endoscope 16. Deployment of a magnetic device 18 is generally illustrated in FIG. 3. As shown, exemplary magnetic anastomosis devices 18 may be delivered through an endoscope 16 such that individual magnet segments self-assemble into a larger magnetic structure—in this particular case, an octagon. When used with the techniques described herein, the devices 18 allow for the delivery of a larger magnetic structures than would otherwise be possible via a small delivery conduit, such as in a standard endoscope, if the devices were deployed as a completed assembly. Larger magnet structures, in turn, allow for the creation of larger anastomoses that are more robust, and achieve greater surgical success. Because the magnetic devices are radiopaque and echogenic, the devices can be positioned using fluoroscopy, direct visualization (trans-illumination or tissue indentation), and ultrasound, e.g., endoscopic ultrasound. The devices 18 can also be ornamented with radiopaque paint or other markers to help identify the polarity of the devices during placement.

The magnetic anastomosis devices 18 of the invention generally comprise magnetic segments that can assume a delivery conformation and a deployed configuration. The delivery configuration is typically linear so that the device can be delivered to a tissue via a laparoscopic “keyhole” incision or with delivery via a natural pathway, e.g., via the esophagus, with an endoscope 16 or similar device. Additionally, the delivery conformation is typically somewhat flexible so that the device can be guided through various curves in the body. Once the device is delivered, the device will assume a deployed configuration of the desired shape and size by converting from the delivery configuration to the deployed configuration automatically. The self-conversion from the delivery configuration to the deployment configuration is directed by coupling structures that cause the magnetic segments to move in the desired way without intervention. Exemplary self-assembling magnetic anastomosis devices 18, such as self-closing, self-opening, and the like, are described in U.S. Pat. No. 8,870,898, U.S. Pat. No. 8,870,899, U.S. patent application Ser. No. 14/522,977, filed Oct. 24, 2014, and U.S. patent application Ser. No. 14/805,916, filed Jul. 22, 2015, the contents of each of which are incorporated by reference herein in their entirety.

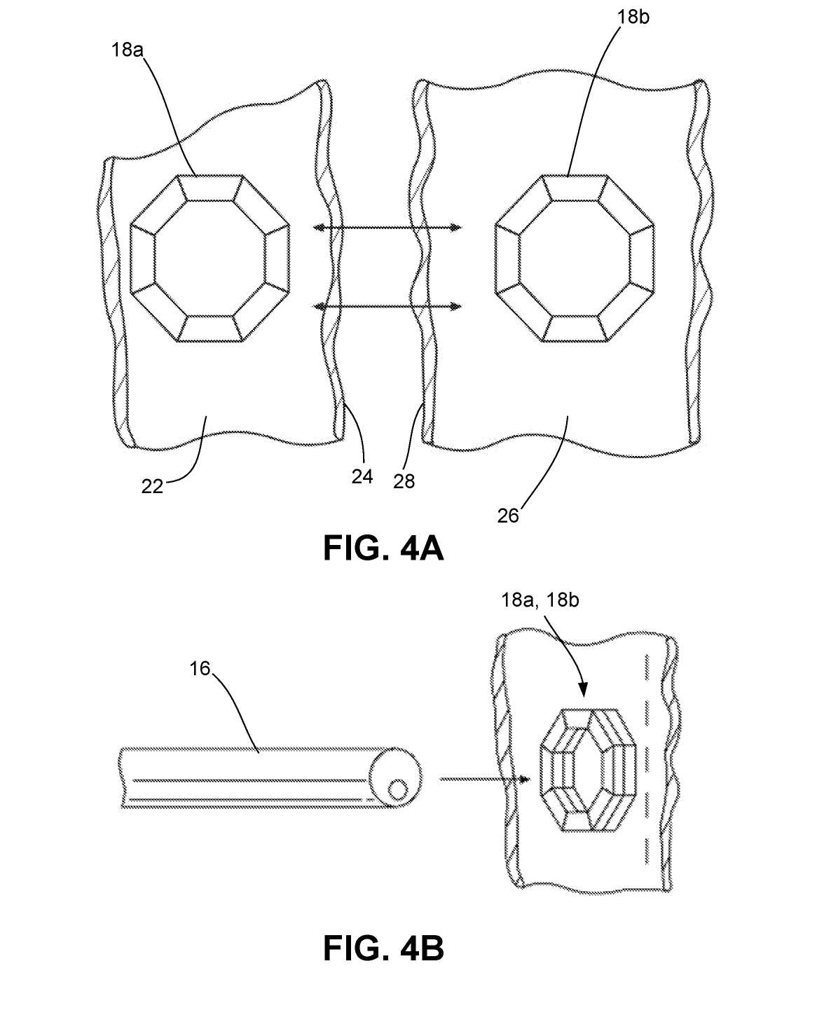

In general, as shown in FIG. 4A, a magnetic anastomosis procedure involves placing a first and a second magnetic structures 18a, 18b adjacent to first and second portions 22, 26 of tissues 24, 28, respectively, thus causing the tissues 24 and 28 to come together. Once the two devices 18a, 18b are brought into proximity, the magnetic structures 18a, 18b mate and bring the tissues 24, 28 together. With time, an anastomosis of the size and shape of the devices 18a, 18b will form and the devices will fall away from the tissue. In particular, the tissues 24, 28 circumscribed by the devices will be allowed to necrose and degrade, providing an opening between the tissues.

Alternatively, because the mated devices 18a and 18b create enough compressive force to stop the blood flow to the tissues 24, 28 trapped between the devices, a surgeon may create an anastomosis by making an incision in the tissues 24, 28 circumscribed by the devices, as shown in FIG. 4B.

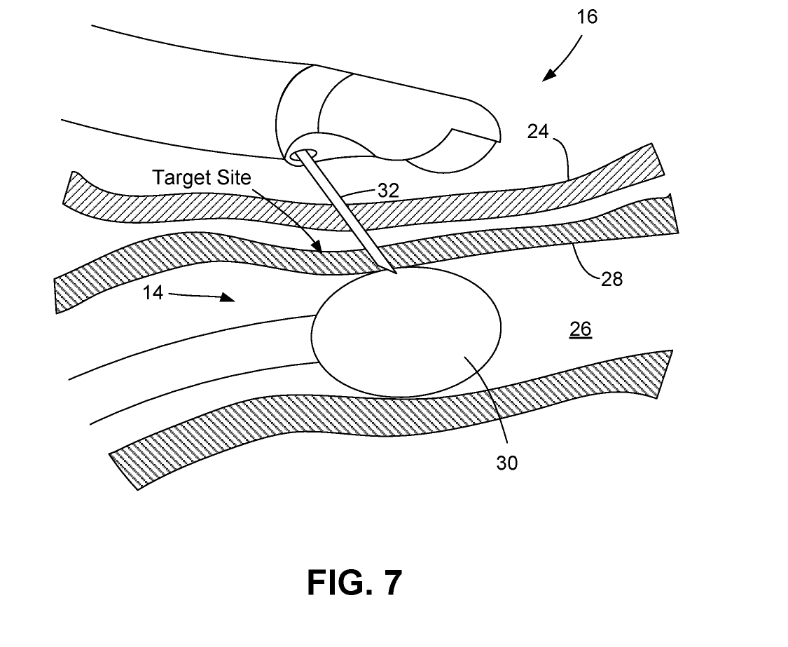

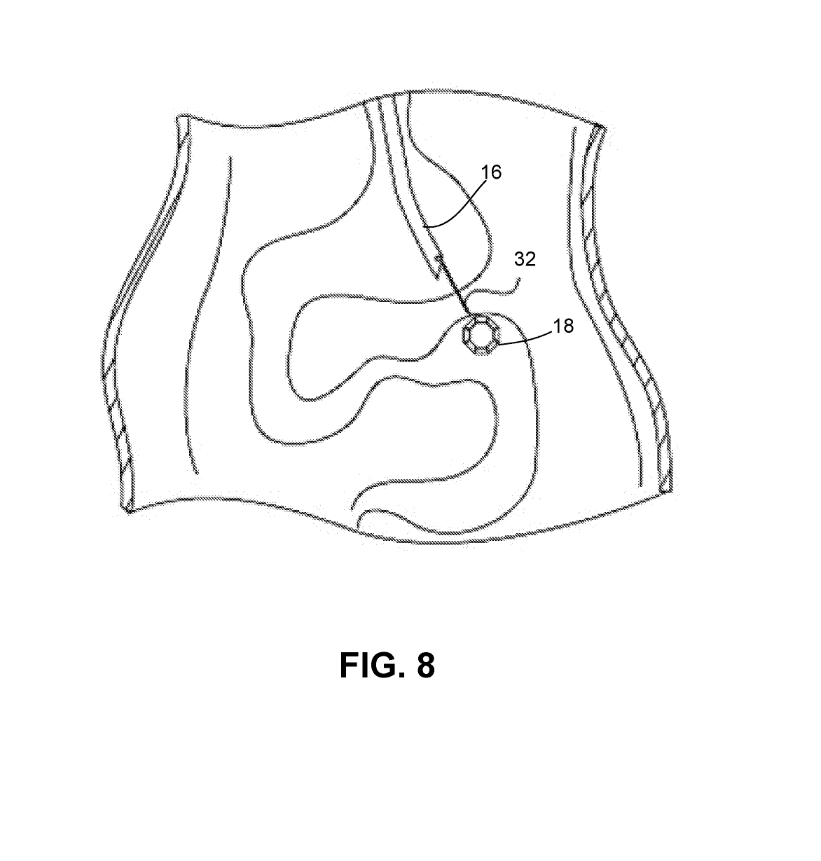

In yet another embodiment, as will be described in greater detail herein, and shown in FIGS. 7 and 8, a surgeon may first cut into, or pierce, the tissues 24, 28, and then deliver device 18b into a portion 26 of the hollow body so as to place device 18b around the incision on tissue 28. The surgeon may then place device 18a into portion 22 of the hollow body so as to deliver device 18a around the incision on tissue 24, and then allow the devices 18a and 18b to couple to one another, so that the devices 18a, 18b circumscribe the incision. As before, once the devices 18a, 18b mate, the blood flow to the incision is quickly cut off.

While the figures and structures of the disclosure are primarily concerned with annular or polygonal structures, it is to be understood that the delivery and construction techniques described herein can be used to make a variety of deployable magnetic structures. For example, self-assembling magnets can re-assemble into a polygonal structure such as a circle, ellipse, square, hexagon, octagon, decagon, or other geometric structure creating a closed loop. The devices may additionally include handles, suture loops, barbs, and protrusions, as needed to achieve the desired performance and to make delivery (and removal) easier.

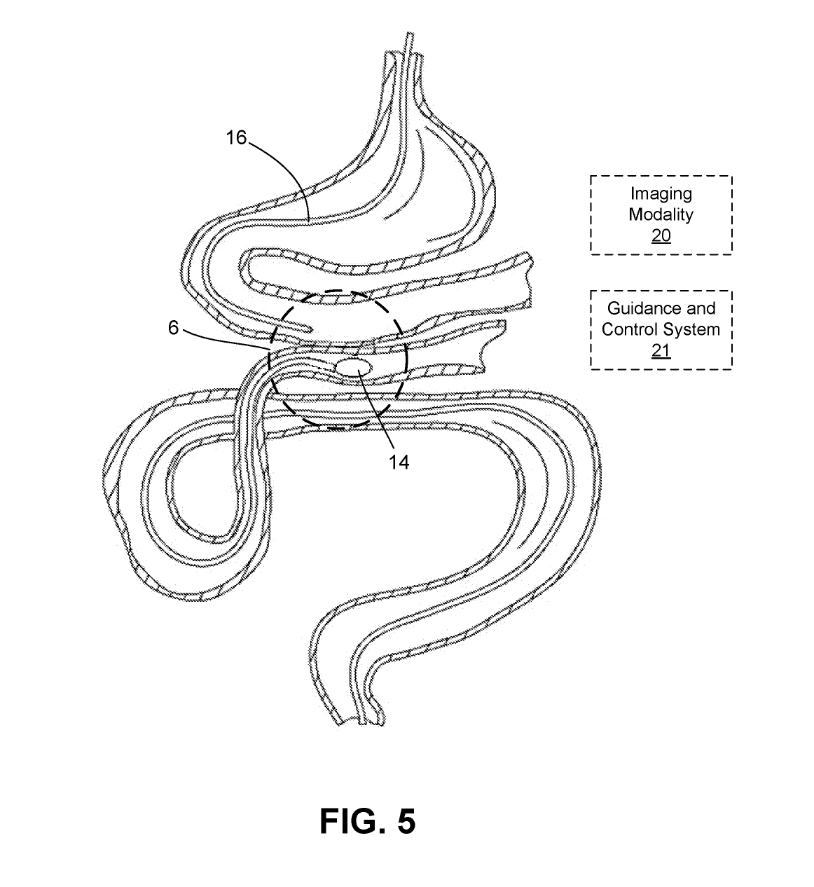

FIG. 5 shows one embodiment of a procedure for forming anastomosis using the targeting system 10 of the present disclosure. As previously described, the system of the present disclosure may be used for forming an anastomosis between first and second portions of a hollow body, such that the targeted member or medium 14 is configured to provide a target site at desired anatomical location within the hollow body for formation of an anastomosis between tissue at a first portion of the hollow body at the target site and tissue at a second portion of the hollow body. As shown in FIG. 5, the hollow body may include the bowel, such that the first portion may be a distal portion of the bowel (e.g., lower small intestine) and the second portion may be a proximal portion of the bowel (e.g., upper small intestine). Accordingly, the single endoscope 16 may be introduced into the upper small intestine via the esophagus and the targeted member or medium 14 may be introduced into the lower small intestine via the colon, for example.

As previously described, the system 10 may utilize an imaging modality 20 configured to provide a visual depiction of the targeted member or medium 14 within the hollow body and the surrounding anatomical location to thereby provide visual depiction of the location of the target site to an operator (e.g., surgeon). For example, during a procedure, the imaging modality 20 is configured to detect the targeted member or medium 14 within the hollow body (e.g., as the targeted member or medium 14 is advancing towards the lower small intestine from the colon) and further provide a visual display of the hollow body and the targeted member or medium 14 within. Once the surgeon reaches a desired position, the surgeon may stop advancement of the targeted member or medium 14 so as to establish the target site at the desired location. Accordingly, the imaging modality 20 can continue to display the location of the targeted member or medium 14 so as to illustrate the targeting site so as to further assist the surgeon in placement of the endoscope 16.

The targeted member or medium 14 may include a contrast material or agent configured to enhance the contrast of the targeting medium relative to surrounding tissue of the hollow body when viewed under a medical imaging procedure provided by the imaging modality 20 to thereby enhance the visibility of the target site. The medical imaging procedure may include, but is not limited to, ultrasound (US), wavelength detection, X-ray-based imaging, illumination, computed tomography (CT), radiography, and fluoroscopy, or a combination thereof. Accordingly, the contrast material or agent may include radiopaque material or radiocontrast medium or agent, respectively. In some embodiments, the contrast material or agent may include a fluorescent tracer material, such as fluorescein, for example and the wavelength emission of fluorescein may be sensed by the imaging modality 20.

As previously described herein, the guidance and control system 21 may be used to combine input obtained from both the endoscope 16 and the imaging modality 20 in order to produce more accurate images or illustrations of the targeted member or medium 14 and endoscope 16 relative to one another and the surrounding tissue. In certain embodiments, with apparatuses and methods of the invention, the input data may be used in combination with one another to construct a three dimensional image of an inside of the hollow body. In particular, image and sensing data may be co-registered with one another to produce images that are more accurate than previously constructed images from the endoscope 16 input and the imaging modality 20 input alone because constructed images of the invention account for distortions. Co-registration may generally refer to any method of re-aligning images, and in particular aligning or overlaying images from different modalities.

An exemplary method of co-registration consistent with the present invention is now described which uses intravascular ultrasound and shape sensing to obtain a co-registered intravascular data set. The invention, however, encompasses any and all intravascular imaging modalities, including without limitation, intravascular ultrasound (IVUS), optical coherence tomography (OCT), external ultrasound, x-ray angiography, Computerized Tomography (CT) angiography, and Magnetic Resonance (MR) angiography. Such modalities can be used instead of intravascular ultrasound and shape sensing modalities and also in addition to such modalities. Any number of modalities is useful for co-registration. Furthermore, modalities suitable for co-registration include functional measurement parameters, in addition, or alternatively, to the shape sensing data, including vessel flow, vessel pressure, FFR, iFR, CFR, etc.

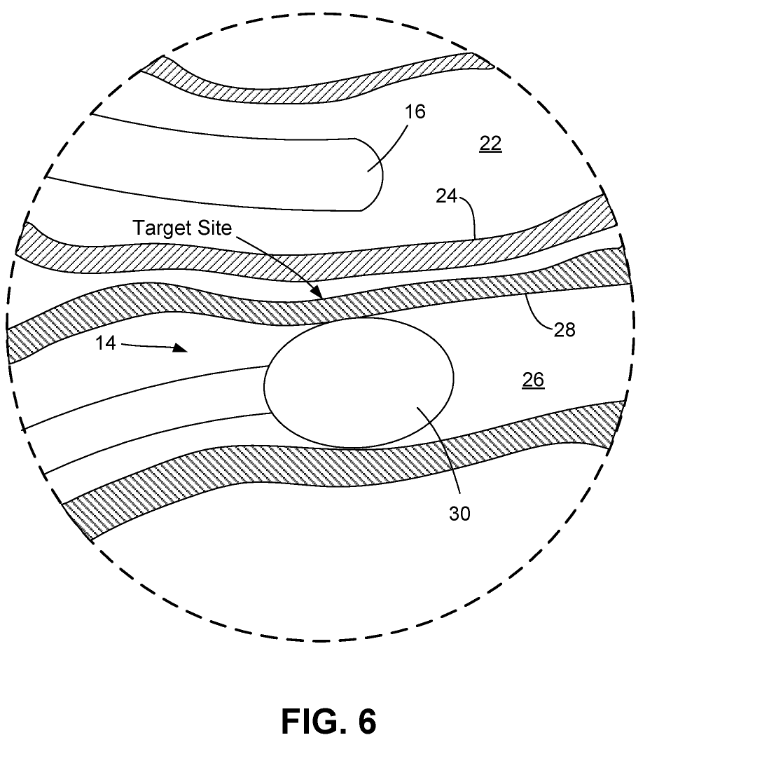

In some embodiments, the targeted member 14 may include a balloon catheter, as illustrated in greater detail in FIG. 6. As shown, the balloon member 30 of the balloon catheter can be expanded at the desired anatomical location within the lower small intestine so as to provide a target site indicating where the anastomosis should be formed. In some embodiments, the balloon member may include a radiocontrast medium configured to enhance the contrast of the expanded balloon member 30 relative to surrounding tissue of the hollow body when viewed under an imaging procedure of the imaging modality 20.

In some embodiments, the balloon member 30 may include a probe configured to emit a signal, including, but not limited to, radio waves, infrared radiation, visible light, or other communication. The imaging modality 20, or the endoscope 16, may be configured to detect such emissions so as to further provide indication as to the location balloon member 30, thereby providing an indication of the target site. For example, in some embodiments, the endoscope 16 may include a detection system configured to detect one or more signals emitted from the probe of the balloon member 30.

For example, in the event that the balloon member 30 includes a contrast material or agent, such as a fluorescent tracer material (e.g., fluorescein), the detection system of the endoscope 16 may include a wavelength detection means configured to detect the wavelength emission of the fluorescein, thereby providing indication to a surgeon that the endoscope 16 is within the desired target site and placement of the magnetic anastomosis devices 18 can begin. In other embodiments, the detection system may be configured to detect the probe emissions (e.g., detect the radio waves, infrared radiation, visible light, high intensity light, ultrasound, etc.).

Accordingly, buy utilizing the imaging modality 20 and/or detection system of the scope 16, the surgeon may advance the scope 16 into the upper small intestine from the esophagus until the scope 16 reaches a portion 22 of the intestine in which tissue 24 is adjacent to the tissue 28 corresponding to the target site. It should be noted that the surgeon may further utilize ultrasound or visualization through the scope 16 to further assist in locating the target site and the position of the scope 16.

The balloon member 30, when expanded at the desired anatomical location, may further serve as an anchor so as to assist in the placement of the magnetic anastomosis devices 18. For example, as shown in FIG. 7, the endoscope 16 is configured to provide access between the different portions 22 and 26 of the upper and lower small intestines, respectively, by way of a needle 32. Upon reaching a tissue 24 adjacent to the target site of tissue 28, the surgeon is able to advance the needle 32 through the tissues 24, 28, so as to pierce the tissues 24, 28 and allow for the delivery and deployment of the magnetic devices 18a, 18b. When piercing through the tissues, the tip of the needle 32 may generally make contact with the balloon member 30. Accordingly, the balloon member 30 may provide resistance upon contact with the needle tip, thereby further providing tactile feedback to the surgeon that the needle puncture is in the correct location (i.e., at the target site), thereby providing confirmation to the surgeon that anastomosis will occur at the desired location (e.g., the target site).

As shown in FIG. 8, the needle 32 may then be used to deliver the first magnetic anastomosis device 18a into the lower small intestine (through the puncture), which is then followed by deployment to of a second magnetic device 18b into the upper small intestine at a location on the tissue adjacent to the target site. It should be noted that the delivery can be guided with fluoroscopy or endoscopic ultrasound. Following self-assembly, these small intestine magnetic devices 18a, 18b couple to one another (e.g., magnetically attracted to one another) through a defined tissue area of the combined thickness of a wall of the tissues at the target site and exert compressive forces on the defined area to form the anastomosis.

While some previously described embodiments of the present invention include the use of a single scope (i.e., scope 16) for the placement of the magnetic anastomosis devices, some embodiments of the present disclosure utilize the two endoscopes, in which a first scope may include the targeted member 14 and the second scope may be embodied as scope 16, and the guidance and control system 21 is further configured to provide automated, or semi-automated, control over the movement of the first and second scopes relative to one another during the procedure. In particular, the guidance and control system may generally include a control platform configured to provide automated control over either of the first and second scopes, or both scopes in unison with one another, based, at least in part on, visual data from the imaging modality 20, emission data from the targeted member 14 (e.g., radio waves, infrared radiation, visible light, or other communication, etc.), and feedback data from one or more sensors of the first and second scopes.

For example, the first and second scopes may be embodied as robotic endoscopes (also referred to herein as “robotic scopes”). The first and second scopes may further include imaging devices and/or sensors for providing feedback to the imaging modality 20 and the guidance and control system 21, wherein the feedback is used to determine the anatomy of inside of the hollow body in which each scope is placed and further to determine the position of the tips of the scopes relative to one another (useful the placement of the magnetic anastomosis devices). The guidance and control system 21 is configured to receive the feedback from the scopes and, in combination with other input data (e.g., visual data from the imaging modality 20, emission data from the targeted member 14, etc.), the guidance and control system 21 is configured to provide automated movement of the first and second scopes relative to one another such that they meet at a defined target site (desired anatomical location within the hollow body for formation of an anastomosis between a first portion of tissue of the hollow body at the target site and a second portion of tissue of the hollow body). Accordingly, based on the data received, the guidance and control system 21 is configured to assist in movement (either in an automated fashion or user-assisted fashion) of either of the first and second scope through the hollow body with precision and accuracy. The guidance and control system 21 may further allow for user intervention (i.e., switch over to fully manual control) at any point during the procedure.

The guidance and control system 21 may be configured to control the deployment and positioning of the magnetic anastomosis device(s). For example, devices and tools within the working channels of the scopes may be automatically advanced, retracted, rotated, measured, and actuated via the guidance and control system 21, either automatically or with manual control.

The first and second scopes may generally include an up to 6 Degree of Freedom (DoF) tip movement, measurement, and control relative to a fixed and known starting point and relative to each other. The scopes may further include a haptic feedback mechanism configured to provide a user with haptic feedback when the scope tip contacts tissue wall and/or the user attempts to penetrate tissue wall(s) for placement of the magnetic anastomosis device(s) scope. For example, the scope tip may include a pressure sensor or strain sensor, for example, configured to provide feedback to the guidance and control system 21. If the sensed strain or pressure is above a predefined threshold, haptic feedback in the form of a physical sensation will be provided to the user via the control mechanism (e.g., handheld controller). Furthermore, the system may be configured to provide an audible signal or alert to the user of the high force being placed on the tissue, thereby placing the user on notice to correct the movement and position of the scope. Accordingly, during user-controlled (or manual) movement of the first or second scopes, the haptic feedback will provide the user with a physical sense of when the probe is in contact with tissue wall and if the user is placing too much pressure or force on the tissue wall as the scope is moving. The haptic feedback data may further be processed by the guidance and control system 21 so as to ensure that the scope movement is either maintained or corrected to avoid undesired contact with tissue wall(s), which may further be useful during automated control of the scopes.

During a procedure including the first and second scopes, specifically during advancement of the scopes within a bowel, for example, feedback data may be collected from the scope itself, in which the scope may include a position sensor on or near the tip of the scope, wherein position of the scope tip in any direction and along the shaft of the scope may be based, at least in part on, movement of the position sensor relative to a known point (e.g., entry point, target site point, etc.). Scope tip position may be known by a sensor on the tip (e.g. EM sensor) but also known by the kinematics (measured joints) of the robot mechanism. Movement and positioning of the scopes may also be determined based on the visual data provided by imaging device(s) on the scope, such as, but not limited to, image capture (camera), ultrasound (US), wavelength detection, X-ray-based imaging, illumination, computed tomography (CT), radiography, and fluoroscopy, or a combination thereof, as well as emission data from the targeted member 14.

As the first and second scopes move closer to the target site, the guidance and control system 21 may be configured to determine when the scope tips are within a defined proximity (e.g., 5 cm or closer) to one another based on feedback data from each of the scopes. For example, each of the scopes may have tracking sensors configured to sense position of one another. In other words, the first scope tracking sensor and second scope tracking sensor are configured to sense proximity to one another and provide the proximity data to the guidance and control system 21. In addition, if the two scopes are registered to each other at the beginning of the procedure, then the respective tip positions will be know relative to each other once the scopes are received inside the respective lumens. In turn, either scope can be controlled via the system 21 to be better positioned for deployment of the magnetic anastomosis device based on the proximity data. For example, once within a defined proximity with one another, the guidance and control system 21 may be configured to independently control movement/articulation of the tip of one of the scopes, or tips of both scopes, such that the tips align with one another at the target site and tissue wall(s) can be slightly compressed between the tips. Again, the haptic feedback provides an alert or signal to the user if the force applied to the tissue falls above a tolerable level and thus ensures that the tissue wall is not prematurely punctured or damaged. Generally, the basic automated movement and positioning mode may include that the guidance and control system 21 automatically moves the first and second scopes to follow the bowel paths up to a certain point, and then, once with in a given range, the system 21 is configured to automatically (or turn over to manual control) for the movement of the scope tips (or distal portions of the scopes) together.

The guidance and control system 21 may further be configured to control deployment and placement of the magnetic anastomosis devices at the target site. In particular, the specific geometry data of the magnetic anastomosis devices may be known, including, not only the dimensions (e.g., diameter, perimeter, height, etc.), but also the initial delivery configuration (e.g., linear self-closing configuration, compressed self-opening configuration, etc.) and the final deployed configuration (e.g., four-sided polygon, six-sided polygon, eight sided polygon, etc.), as well as the magnetic configuration (e.g., north and south pole pattern). Accordingly, based, at least in part, on the geometry data of the device, the guidance and control system 21 is configured to automatically deliver and position the magnetic anastomosis device with accuracy and precision.

For example, the system 21 may begin deployment of the magnetic anastomosis device from the working channel of the scope, wherein the deployment may generally begin with penetration of the tissue walls with a needle or other piercing element. In some embodiments, the robotic scope may be equipped with an imaging device (i.e., camera or other imaging modality, such as ultrasound) configured to obtain imaging data, such that the target site is visible and any critical information regarding the condition of the target site (e.g., state of tissue and whether the target site is clear of debris or other obstacles) can be determined based on the robotic scope's imaging data. Additionally, or alternatively, imaging data from the room or patient-based imaging modality may be used to determine the condition of the target site. If the image data shows a structure or other obstacle in the path of the needle, advancement of the needle can be completely halted and the scope's tip position can be adjusted to alter the path of the needle to avoid the structure while still maintaining adequate alignment with the target site, ensuring that the altered needle path will still engage and penetrate the target site.

Once a magnetic anastomosis device is fully deployed from the working channel of the scope, the system 21 is able to automatically position a first magnetic anastomosis device relative to a second magnetic anastomosis device, based, at least in part, on the known geometry data of each device and the known scope tip positions. It should be noted that the system 21 may further provide a combination of automated and manual-assist for the deployment and positioning. In some embodiments, the magnetic anastomosis devices may include sensors or the like which provide feedback indicating the placement of the device relative to one another whether a sufficient symmetrical coupling between the magnetic anastomosis devices has been achieved, as described in U.S. application Ser. No. 15/150,397, filed May 9, 2016, the content of which is incorporated by reference herein in its entirety.

As understood herein, rather than using two scopes for the placement of magnetic anastomosis devices at a desired target site, the systems of the present disclosure allow for the use of a single endoscope (e.g., robotic scope) for the placement of multiple magnetic anastomosis devices. For example, in some embodiments, the robotic scope may include two or more working channels, thereby allowing for independent delivery of two or more magnetic anastomosis devices at the desired target site. Additionally, or alternatively, the one or more working channels may have a large enough diameter (e.g., 6 to 7 millimeters) to accommodate different delivery configurations of the magnetic anastomosis devices, such as the linear self-closing configuration, the compressed self-opening configuration, and any additional components involved in the delivery, such as a guidewire or guide elements (e.g., sutures coupled to magnetic segments), for example. Furthermore, the magnetic anastomosis device may be delivered as a collar around the distal end of the scope, such that, release of the device when deployed results in the device remaining co-linear with the scope.

When performing the procedure with the single robotic scope, the targeted member or medium is still relied upon to provide a target site at a desired anatomical location within a hollow body for formation of the anastomosis between a first portion of tissue of the hollow body and a second portion of tissue of the hollow body. Accordingly, as previously described, the targeted member or medium can be provided into the hollow organ via a catheter, scope, wire, or may further be administered orally, generally in the form of a pill or medium that is detectable by scintigraphy, fluoroscopy, or patient-based imaging modality.

Furthermore, the system of the present disclosure may include multiple imaging modalities for the automated, or semi-automated movement of the robotic scope and subsequent deployment of the multiple magnetic anastomosis devices. For example, one type of imaging modality could include a room-based system configured to track the robotic scopes movements based on one or more sensors (e.g., electromagnetic or other sensors) on the robotic scope, such as sensors placed on the tip of the robotic scope or other portions of the robotic scope shaft. Additionally, or alternatively, the imaging modality may also include a patient-based system that uses fluoroscopy, CT, and/or ultrasound for imaging of the patient, including imaging of the GI tract. Additionally, or alternatively, the imaging modality may include imaging devices provided on the robotic scope itself, as previously described herein.

In a similar manner as previously described herein, during the procedure, the robotic scope is inserted within the hollow body organ and is able to locate (i.e., move into position with) the targeted member or medium, either relying on the robotic scope's own imaging modality, and/or based on direction from the room-based and/or patient-based imaging modalities. For example, the robotic scope tip could be registered between the robotic scope shaft and the other imaging modalities (room-based and/or patient-based). Then, the targeted member or medium position (as imaged by the room or patient source) could be known relative to the robotic scope tip. Once the robotic scope tip is co-located relative to the targeted member or medium, the access device (i.e., needle) can be deployed to go from the scope lumen (lumen in which the robotic scope is positioned) to the target lumen (lumen in which the targeted member or medium is positioned). As understood, the access device could be a needle, cautery, or other device that penetrates both lumens and then makes a connection between the tissue walls of the two lumens. A first magnetic anastomosis device may then be deployed within the targeted section, upon which the first device is pulled toward the scope lumen, and a second magnetic anastomosis device may be deployed within the scope lumen and then mated with (by way of magnetic attraction) to the first device within the target lumen, thereby compressing the tissue walls of the lumens together to being the anastomosis process.

Accordingly, the targeting system of the present disclosure provides improved placement of magnetic compression devices at a desired target site so as to create accurate anastomoses between tissues. By providing a targeted member or medium, an operator can better visualize the placement of magnetic compression devices within one or more hollow bodies of a patient and further ensure that such placement is more accurate, leading to anastomosis formation in the desired location, cutting down on repeat procedures. Furthermore, placement of the magnetic devices is achieved by way of a single scope, rather than having to work with two separate scopes, thereby overcoming the challenges encountered when dealing with two or more scope devices and further requiring less towers to support the additional scope(s) or components, decreasing costs and space required.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein

What is claimed is:

1. A system for forming an anastomosis between tissues within a patient, the system comprising:
a targeted member or medium configured to be placed within a hollow body of a patient, the targeted member or medium configured to provide a target site at desired anatomical location within the hollow body for formation of an anastomosis between a first portion of tissue of the hollow body at the target site and a second portion of tissue of the hollow body or an adjacent tissue of a second hollow body; and
an access device configured to provide access between the first and second portions of tissue of the hollow body, or between the first portion of tissue of the hollow body and an adjacent tissue of the second hollow body, the access device configured to deliver and position first and second implantable magnetic anastomosis devices relative to the first and second portions of tissue or adjacent tissue for the formation of an anastomosis between tissues, the first implantable magnetic anastomosis device to be placed within the hollow body at the first portion of tissue associated with the target site and the second implantable magnetic anastomosis device to be placed either within the hollow body at the second portion of tissue corresponding to the target site, or within the second hollow body at a position on the adjacent tissue corresponding to the target site.

2. The system of claim 1, wherein the first and second implantable magnetic anastomosis devices are configured to be magnetically attracted to one another through a defined tissue area of the combined thickness of a wall of the tissues at the target site and exert compressive forces on the defined area to form the anastomosis.

3. The system of claim 1, wherein the targeted member or medium comprises a contrast material or agent configured to enhance the contrast of the targeted medium relative to surrounding tissue of the hollow body when viewed under a medical imaging procedure based on detection of the targeted member or medium.

4. The system of claim 3, the system further comprising an imaging modality configured to provide a visual depiction of the targeted member or medium within the hollow body and the surrounding anatomical location to thereby provide visual depiction of the location of the target site to an operator.

5. The system of claim 4, wherein the detection of the targeted member or medium is provided by the imaging modality to thereby enhance the visibility of the target site.

6. The system of claim 3, the system further comprising a detection system configured to detect the targeted member or medium to thereby enhance the visibility of the target site.

7. The system of claim 3, wherein the medical imaging procedure is at least one of ultrasound (US), wavelength detection, X-ray-based imaging, illumination, computed tomography (CT), radiography, and fluoroscopy.

8. The system of claim 7, wherein the contrast material or agent comprises a radiopaque material or radiocontrast medium or agent, respectively.

9. The system of claim 7, wherein the contrast material or agent comprises a fluorescent tracer material.

10. The system of claim 9, wherein the fluorescent tracer material is fluorescein.

11. The system of claim 10, further comprising a detection system configured to detect the targeted member or medium to thereby enhance the visibility of the target site, wherein the detection system comprises a wavelength detection means configured to detect the wavelength emission of the fluorescein.

12. The system of claim 1, wherein the targeted member comprises a probe configured emit at least one of radio waves, infrared radiation, and visible light.

13. The system of claim 12, wherein the detection system of the access device is configured to detect the probe emissions.

14. The system of claim 1, wherein the targeted member comprises a catheter configured to be introduced into the hollow body, the catheter comprises an expandable balloon member configured to provide the target site.

15. The system of claim 14, wherein the balloon member comprises a radiopaque material configured to enhance the contrast of the balloon member relative to surrounding tissue of the hollow body when viewed under an imaging procedure.

16. The system of claim 14, wherein the balloon member is configured to receive a radiocontrast medium to thereby cause the balloon member to expand and exert a force upon the desired anatomical location within the hollow body, the radiocontrast medium is configured to enhance the contrast of the expanded balloon member relative to surrounding tissue of the hollow body when viewed under an imaging procedure.

17. The system of claim 1, wherein the access device comprises an endoscope configured to be introduced into the hollow body or the second hollow body, the endoscope having a working channel for allowing deployment of the first and second implantable magnetic anastomosis devices from the endoscope into the associated portions of tissue.

18. The system of claim 17, further comprising a delivery needle having a distal tip configured to puncture through the walls of the tissues and further deliver the first implantable magnetic anastomosis device into the hollow body at the first portion of tissue at the target site.

19. The system of claim 1, wherein each of the first and second implantable magnetic anastomosis devices comprises:
an assembly of a plurality of magnetic segments; and
an exterior guiding member coupled to at least two magnetic segments, wherein the exterior guiding member directs the plurality of magnetic segments to reconfigure from a delivery configuration to a deployed configuration.

20. The system of claim 19, wherein, when in the delivery configuration, the assembly of the plurality of magnetic segments is sized to fit within a working channel of the access device, and, when in the deployed configuration, the assembly of the plurality of magnetic segments is a polygon.

21. The system of claim 19, wherein the exterior guiding member comprises an exoskeleton that covers a portion of the exterior of the at least two magnetic segments and is configured to direct the magnetic segments to self-assemble from the delivery configuration to the deployed configuration, wherein the deployed configuration is a polygon.

22. The system of claim 1, wherein the tissues comprise a portion of at least one of the stomach, gallbladder, pancreas, duodenum, small intestine, large intestine, and bowel.

23. The system of claim 22, wherein hollow body is the bowel of the patient, and the first portion is a distal portion of the bowel and the second portion is a proximal portion of the bowel.

24. The system of claim 23, wherein the targeted member or medium is configured to be introduced into the distal portion of the bowel via the colon and the access device is configured to be introduced into the proximal portion of the bowel via the esophagus.

25. The system of claim 24, wherein the anastomosis is formed between the proximal and distal portions of the bowel and create a portion of partially bypassed bowel configured to continue its native physiological function.
